(19) United States
(12) Patent Application Publication
(10) Pub. No.: US 2004/0116777 A1
(43) Pub. Date: Jun. 17, 2004

(54) Guided retractor and methods of use

(76) Inventors: Jeffrey Larson, Spokane, WA (US); Scott Schorer, Niwot, CO (US); Theodore Bertele, Longmont, CO (US); Louis Greenberg, Boulder, CO (US)

Correspondence Address: ROBERT D. FISH; RUTAN & TUCKER, LLP, P.O. BOX 1950, 611 ANTON BLVD., 14TH FLOOR, COSTA MESA, CA, 92628-1950, US

(21) Appl. No.: 10/645,136

(22) Filed: Aug. 20, 2003

Related U.S. Application Data
(60) Provisional application No. 60/433,343, filed on Dec. 13, 2002.

(51) Int. Cl. A61B 17/56

(52) U.S. Cl. 600/210

(57) ABSTRACT

The present invention provides methods and apparatus in which a surgical retractor comprises a plurality of mechanically coupled tissue retaining walls that are guided into position along one or more guides previously implanted into the patient. The walls are preferably coupled by pivots, so that separating some of the walls from one another opens an operating space. There are preferably two guides, which are driven or screwed into the pedicles of vertebrae, or other bone. Since practical considerations will usually mean that the guides are completely parallel to one another, the retractor has oversized channels to receive the guides. The channels are best disposed in a frame, which also serves to hold lock the walls apart. Various convenience features are contemplated including a web disposed between the walls, which expands as the walls are separated. Also contemplated are projections from near the bottoms of one or more of the walls, which can alternatively or additionally help to hold the underlying tissue in place.

This application claims priority to provisional application No. 60/433,343, filed on Dec. 13, 2002.

### FIELD OF THE INVENTION

The field of the invention is surgical retractors.

### BACKGROUND

Many types of surgical retractors are known. The simplest devices are tubular probes, or probes adapted with a paddle or other somewhat flatter surface. Recent embodiments of that concept are depicted in U.S. Pat. No. 6,206,826 to Mathews et al. (March 2001). More complicated retractors utilize scissors, bow string, or screw-jack expanders that operate against mating paddles. Those retractors have the advantage of being able to lock the paddles in place, leaving at least one of the surgeon's hands free for other actions. See e.g., U.S. Pat. No. 6,471,644 to Sidor (October 2002). Still other retractors are self opening, including Cosgrove et al., U.S. Pat. No 6,162,172 (December 2000). All cited patents herein are incorporated herein by reference.

While undoubtedly useful in many respects, none of the above-mentioned retractors are readily fixed in position relative to one or more bones. U.S. Pat. No. 5,027,793 to Engelhardt et al. (July 1991) addresses that need to some extent, by providing spikes on the bottom of a retractor wall, and further providing spikes that can be driven into the bone. The contemplated use is to resect the operating area down to the bone, position the retractor, and then pound both the retractor and the spikes into place.

A problem remains, however, in that the resection required to properly position the retractor can cause considerable trauma to the overlying and surrounding tissues. Another problem is that multiple retractors are needed to retain tissue pushing into the operating area from different directions. The Engelhardt et al. retractor, for example, did not have to address that issue because the preferred application was acetabular surgery, in which the major encroachment was from gluteus muscles that are all substantially superior to the operating site.

In spinal and some other surgeries these problems can be especially severe. Thus, there is still a need to provide methods and apparatus in which an operating space can be positioned and opened with respect to specific areas of bone, while reducing trauma to surrounding tissue.

### SUMMARY OF THE INVENTION

To that end the present invention provides methods and apparatus in which a surgical retractor comprises a plurality of mechanically coupled tissue retaining walls, which are guided into position along one or more guides previously implanted into the patient.

Preferred embodiments utilize two main walls, and four smaller walls, one on each of the ends of the two main walls. In such embodiments all of the walls are coupled by pivots, such that the faces of the two main walls can be moved towards or apart from each other to open or close an operating space. The faces of at least the main walls are preferably flat, but can be any other suitable shape, including convex. The invention is particularly suited for operating on or near curved bony surfaces, and the bottoms of the walls can be compliant (i.e., advantageously adapted to fit and/or conform to the bone surface below).

There are preferably two guides, which are driven or screwed into the pedicles of vertebrae, or other bone. The various guides can be implanted into different bones, or different areas of the same bone. Since practical considerations will usually mean that the guides are parallel to one another, the retractor has oversized channels to receive the guides. The channels can be circular in cross section, but are more preferably elongated into an oblong or other slotted shape.

The channels are best disposed in a frame, which also serves to hold lock the walls apart. Any suitable devices can be used to move apart the main walls to open the operating space, including for example a simple wedge or T-bar, or a mechanism disposed on the frame. The frame can be held in place relative to the guides by wires, nuts, clamps, and so forth..

Various convenience features are contemplated including a web disposed between the walls, which expands as the walls are separated. The web can be cut, torn, bent away, or otherwise manipulated to expose the tissue below. Also contemplated are projections from near the bottoms of one or more of the walls, which can alternatively or additionally help to hold the underlying tissue in place, and can similarly be removed in any suitable manner from the corresponding wall. The frame or other portion of the retractor can be transparent to aid in surgeon visualization.

Various objects, features, aspects and advantages of the present invention will become more apparent from the following detailed description of preferred embodiments of the invention, along with the accompanying drawings in which like numerals represent like components.

### BRIEF DESCRIPTION OF THE DRAWING

FIG. 1 is a perspective view of a retractor according to the inventive subject matter, in an open configuration.

FIG. 2 is a perspective view of the retractor of FIG. 1, disposed in a closed configuration.

FIG. 3 is a perspective view of the back and spine of a patient, in which finger dissection is being employed to locate a pedicle of a vertebra.

FIG. 4 is a horizontal cross-sectional view of a vertebra, showing use of an awl to punch a guide hole into a pedicle.

FIG. 5 is a horizontal cross-sectional view of the vertebra of FIG. 4, in which a screw is being screwed into the hole created in FIG. 4.

FIG. 6 is a perspective view of the back and spine of a patient in which the closed retractor of FIG. 2 is being fitted onto the guides implanted into adjacent vertebrae.

FIG. 7 is a perspective view of the back and spine of the patient of FIG. 6 in which the retractor is being opened by an opening tool.

FIG. 8 is a perspective view of the back and spine of the patient of FIG. 6 in which the retractor has been opened, and the web is being removed to expose various fingers and the underlying tissue.

FIG. 9 is a perspective view of the back and spine of the patient of FIG. 6 in which the retractor has been opened, and various fingers (bottom tissue retainers) are being removed.

### DETAILED DESCRIPTION

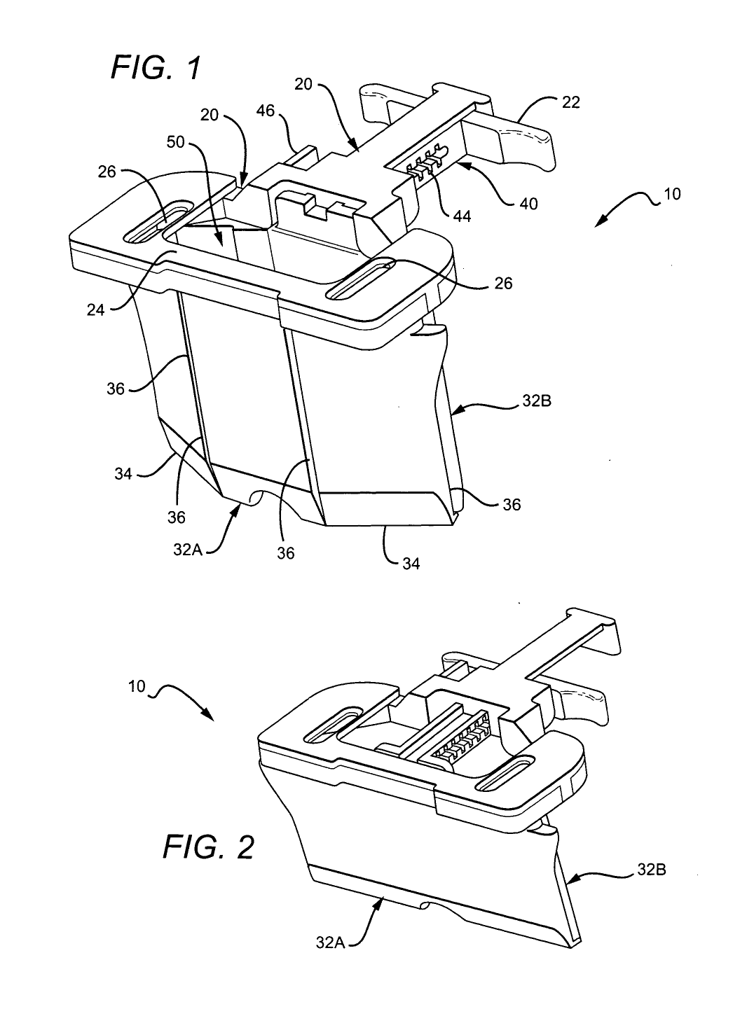

FIG. 1 generally depicts a retractor 10, having a frame 20, major walls 32A, 32B and minor walls 34, and a locking/opening mechanism 40. The various walls 32A, 32B, 34 are coupled by six hinges 36, and in the open position depicted in the figure cooperate to define an operating space 50.

The frame 20 can be any suitable size and shape according to the particular applications, with larger frames being generally more useful for larger incisions. For posterior lumber surgery on adult humans, the overall dimensions of an especially preferred frame are about 5.5 cm in depth, 3.5 cm in length, 3 cm in width. Frame 20 is preferably made from Delrin®, but can be made of any suitable material, especially a nontoxic polymer such as polyethylene. The frame 20 can advantageously be colored to reduce glare from operating room lighting, and some or all of the frame can be relatively transparent.

Frame 20 generally comprises a handle portion 22 that includes the locking mechanism 40, and a perimeter 24 around the operating space 50. The locking mechanism 40 is shown as a ratchet, but all other suitable locking mechanisms are also contemplated, especially those that provide for a high degree of reliability and ease of operation. At least one of the walls 32A, 32B, 34 is preferably coupled to the perimeter 24 using a pin (not shown).

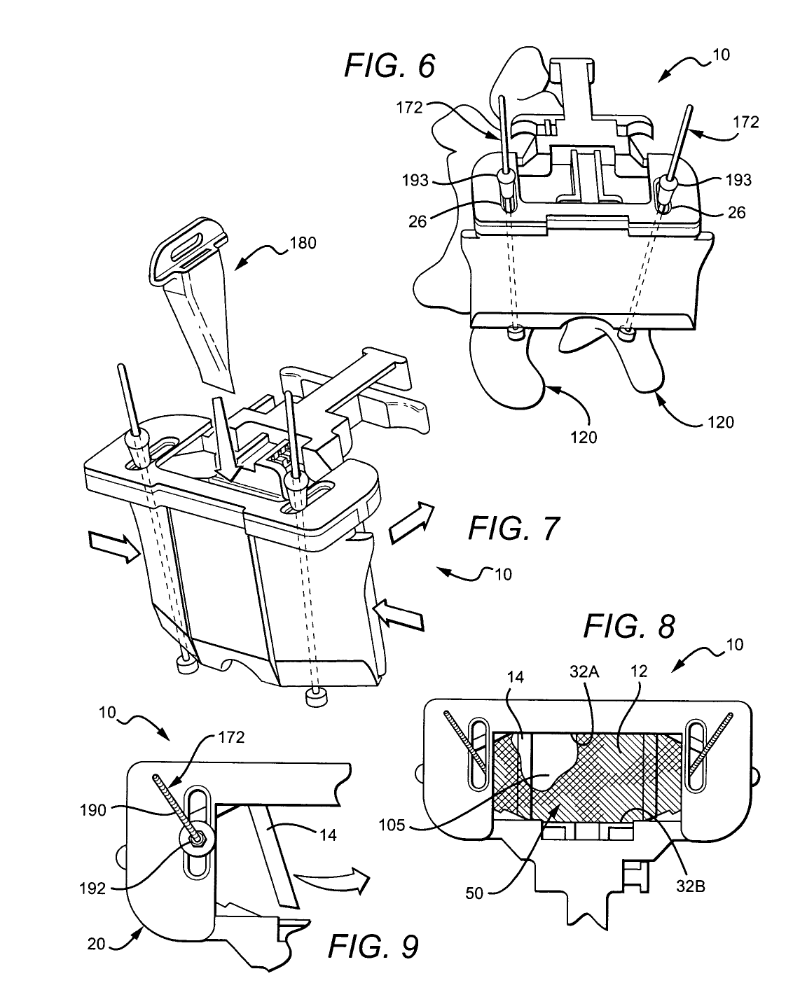

Channels 26 are located on opposite sides of the perimeter 24, and are each sized to receive one of the guides 172 (see FIGS. 4-9). The system is designed to work with a wide range of pedicle screw or other bone fixation systems, and with various numbers of guides, regardless of the specific relationship between screw and guide. In addition, the passageways defined by the channels 26 should be oversized with respect to the outside diameters of the shafts of the guides 172 so that the channels 26 can receive guides 172 that are out of parallel or in some other manner not perfectly aligned with each other. In preferred embodiments the channels define a passageway having a diameter of about 5 to 15 mm, whereas the guides 172 (see FIGS. 5, 6) preferably have a corresponding diameter of about 4 to 6 mm. All ranges set forth herein should be interpreted as inclusive of the endpoints.

As with other components, the various walls 32A, 32B, 34 are preferably made of a biocompatible material, and here again they can have any suitable sizes and shapes, depending on the surgical site or sites for which they are intended. The currently preferred material for retractor walls is polypropylene. Walls 32A, 32B, 34, for example, can be mostly rectangular in vertical cross-section as shown, with bottoms of at least the major walls 32A, 32B curved to accommodate specific bone shapes, such as that of the spinous processes of the vertebrae in spinal surgery. It is also contemplated that the bottoms of at least the major walls 32A, 32B can be pliable, to conform at least partially to projections and depressions of the underling bone. Walls 32A, 32B, 34 are depicted in the figures as having flat sides, but alternatives may be bowed outwardly (convex), inwardly (concave), or may have any other suitable horizontal cross-section.

One or more of the walls (not shown) can even be inflatable, made out of balloons that define the opening. Of course, the walls 32A, 32B, 34 must be sturdy enough, and therefore thick enough, to withstand the expected forces placed upon them. On the other hand the walls 32A, 32B, 34 are preferably not so thin that they would cut into the tissue below during deployment. On the other hand, the walls 32A, 32B, 34 should not be so thick as to significantly interfere with the size of the operating area. Preferred thickness is from about 3.5 mm to about 5 mm at the thickest point, tapering down to a thickness of 1.5 mm-3 mm at the bottom of each wall. The walls can also be nested in any suitable manner, which simply means that a portion of one wall extends around a portion of another wall.

The hinges 36 are shown as continuations of the walls 32A, 32B, 34. Indeed all of the walls and hinges can be molded as a single piece, with each of the hinges 36 being formed as an especially thin edge of a wall. This is effectively a “living hinge” that can handle multiple openings, using material properties of polypropylene. All other suitable configurations of hinges are also contemplated. For example, instead of four minor walls 34, the major walls 32A, 32B could be coupled by only a single outwardly bowed, flexible piece (not shown) at each end. Certainly the total number of walls can be greater or less than 6.

The term “wall” is used herein in a very broad sense, to mean any sort of tissue retaining barrier, generally longer than tall, and considerably taller than thick. Retractor 10 could thus be termed a “linear retractor” to distinguish it from point retractors that are basically pen-shaped probes. But neither the retractor as a whole nor any of the walls are necessarily linear. The term certainly does not require that the wall be so thin as to constitute a cutting blade. Nor does the term “wall” require that the sides thereof be completely patent. The sides of the walls may be pitted or indented as would occur if the sides had a mesh coating (not shown), and the sides may even have through holes (not shown).

Locking/opening mechanism 40 is shown as a typical ratcheting type mechanism, with teeth 44, and having a release 46. Frame 20 can have both a locking mechanism and an opening mechanism (not shown), or either one by itself. There are numerous other locking and/or opening mechanisms known to the field, and presumably others will become known in the future. It is contemplated that any suitable locking and/or opening mechanisms can be used.

Operating space 50 will be larger or smaller depending on the sizes and shapes of the walls, and the extent to which the walls are separated out from one another. Preferred area of the operating space 50 is between 7 cm² and 14 cm².

FIG. 2 generally depicts the retractor 10 of claim 1, disposed in a closed configuration. The terms “closed” and “open” with respect to configurations of the retractor 10 are relative. Thus, closed merely means substantially closed, but does not require complete closure, so that the walls 32A, 32B are juxtaposed. In a closed position the walls 32A, 32B may well be separated by up to 1 mm or more. Similarly, in a contemplated open configuration, walls 32A, 32B would likely be separated by at least 1.5 cm, but may be separated by up to 2.3 cm or more.

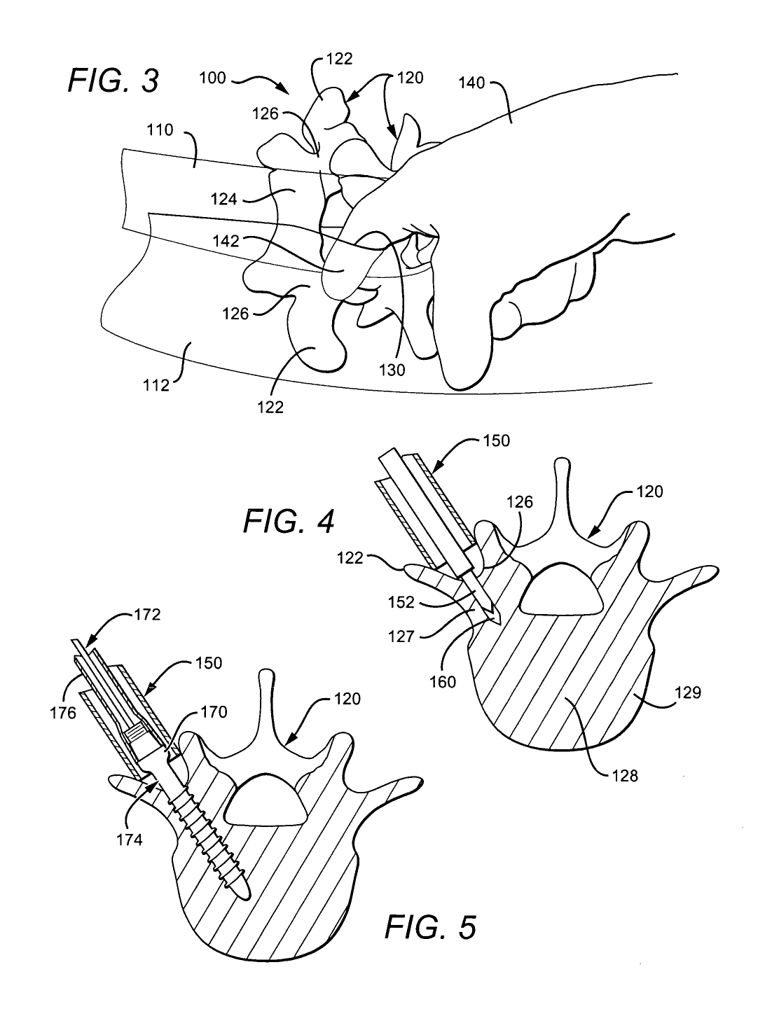

FIG. 3 generally depicts a portion of the spine 100 of a patient, in which the paraspinous muscles are designated schematically by semitransparent bands 110, 112, respectively. The spine 100 includes vertebrae 120, each of which includes transverse processes 122, spinous processes 124, and pedicles 126. An incision 130 has been made, and a finger 142 of hand 140 is being used to dissect through the muscle and locate one of the pedicles 126. Of course a wedge, probe or other tool could be use in place of or in addition to the finger 142 to locate the pedicles.

FIG. 4 generally depicts cannula 150 that positions an awl or probe 152 for use in producing a hole 160 in the pedicle 126. The awl 152 can be manually pushed or otherwise forced through the cortex 127 of the pedicle. Cannula 150 is preferably made of radiolucent material such as plastic or carbon fiber, while awl and probe 152, and other tool attachments and inserts are all preferably made of metal such as surgical steel, titanium, or other durable, radio opaque material. Positioning the cannula 150 can be aided by fluoroscopy or other visualization technique.

In preferred methods, the awl 152 is withdrawn, and a longer, thinner probe (not shown) is inserted through the pedicle 126 into the softer medulla 128 of the body 129 of the vertebra 120. The longer pin is then withdrawn, and in FIG. 5 a screwdriver 176 is used to insert a screw 174. The screw has a head 170, which holds a guide 172 in place. The screwdriver 176 is then removed, leaving the screw 174 implanted into the vertebra 120, and guide 172 rotatably attached to the top of screw 174. The process is repeated to insert another guide 172 into another area of bone, which in the case of spinal surgery is most likely the pedicle of an immediately superior or inferior vertebra on the same side. In other surgeries (not shown), the second, or possibly even a further guide, can be inserted into a different location of the same bone as received the first guide.

In FIG. 6 the guides 172 that are implanted into adjacent vertebrae 120 have been inserted into the channels 26 of the closed retractor 10. Those skilled in the art will realize that the channels can have other configurations besides those shown in the drawing, and can be multi-level rather than simply a single level.

In FIG. 7 the retractor 10 is being opened by an expander 180, which is manually inserted between the opposing walls to produce and widen a gap between them. In this figure the expander generally comprises a wedge with a handle. The expander 180 is preferable over using unassisted fingers because it involves a mechanical advantage. Alternatively, the retractor can be opened using a thumb and fingers-opposing force method using the handle 22 and frame 20. There are numerous alternatives which may or may not involve any mechanical advantage, including for example a T handle coupled to a shaft and a cam (not shown).

In FIG. 8 the retractor 10 has been opened to reveal a web 12 positioned between walls 32A, 32B and 34. The web 12 is preferably a thin, flexible sheet of latex or other biocompatible plastic, which can be easily cut, ripped, or in some other manner disrupted to expose various retaining fingers 14 and the underlying tissue 105. Web 12 is shown as covering the entire floor of the operating space 50, but it could alternatively cover a lesser space, and could extend between or among different walls. The fingers 14 are depicted as extending from or rotating out below the web 12, but some or all of the fingers 14 could alternatively be positioned above the web 12. Each of web 12 and fingers 14 are certainly optional.

In FIG. 9 the retractor 10 has been opened, and various fingers 14 are being removed. Such removal can be accomplished in any suitable manner, including by cutting (as with a scalpel or scissors), bending by hand or with a tool, and so forth. There may be wide fingers, narrow fingers, long or short fingers, closely spaced or widely spaced fingers, flat or rounded fingers, and so on (not shown). Where fingers are used, they are preferably molded as continuous extensions of the walls.

Also shown in FIGS. 8 and 9 are threads 190 the guides 172 can be at least partially threaded, and can thereby that receive wing nuts or other correspondingly threaded pieces 192 that assist in anchoring the frame 20 to the guides 172. In alternative configurations one could use non-threaded lock down pieces such as finger clamps 193. In especially preferred embodiments alternative templates (not shown) can be placed on top of the frame, and held in place using the wing nuts, finger clamps, or other hold-down devices. The frame can also be used to hold additional devices, such as suction or lighting, introduced into the field 50 and held in place by a coupling device on the frame 20.

Preferred methods of inserting a tissue retractor 10 into a patient involve the steps of providing a retractor 10 having paired tissue retracting surfaces (such as on walls 32A, 32B, 34) and first and second guide receiving areas (such as channels 26); percutaneously or otherwise implanting first and second guides (such as guides 172) into different areas of bone in the patient; then positioning upper ends of the first and second guides through the first and second guide receiving areas, respectively, then fully inserting the retractor down the guides and into the patient, effectively splitting the muscle; and finally moving the tissue retracting surfaces apart from one another to open the operating space. These methods are especially useful where one or more of the guides are screws, which are implanted into very specific anatomical structures such as the pedicles of vertebrae. The contemplated methods are also extremely useful in opening operating spaces overlying adjacent bones. Especially preferred methods optionally employ nuts, clamps, or other readily attachable and tightenable mechanisms to stabilize the retractor 10 on the guides.

From the description above, it should now be apparent that the novel methods and apparatus disclosed herein turn the normal retracting procedure on its head. Instead of positioning the retaining wall or walls and then holding them in place by implanting spikes or posts into the bone, the present procedure implants the spikes or posts, and then uses them as guides to position the retaining wall(s).

The advantages of turning the procedure around are significant. Among other things, this new procedure allows the surgeon to exactly position the retractor 10 at the intended operative site because the positioning can be done precisely with respect to underlying bony structures (e.g., the pedicle 126 of a vertebra). The screws are implanted where the surgeon wants them, and the guides 172, being attached to the top of the screws guide the retractor down into the desired anatomy, splitting the muscles, and defining a operating site 50 within the walls 32A, 32B and 34. After that the operating site 50 is opened, giving the surgeon the desired exposure needed to conduct the surgery., without excess retraction and resulting tissue destruction.

Another advantage is that these new methods and apparatus speed up the procedure and makes more efficient use of resources relative to the prior art. Among other things, after the guides 172 and screws 174 are placed and the retractor 10 is attached and opened, there is no more need for fluoroscopy, which can be moved along to a different room.

Still other advantages involve convenience and reduction in surgeon stress. The novel methods and apparatus make it mentally easier on the surgeon. After the screws 174 are in, in the first part of the procedure, everything else in terms of opening the operating site is fairly straightforward. This helps the surgeon relax mentally and physically.

Thus, specific embodiments and applications of novel retractors have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

1. A retractor comprising:
a first tissue retaining wall coupled to a first guide receiving channel; and
a second tissue retaining wall movably coupled to the first tissue retaining wall.

2. The retractor of claim 1, wherein each of the retaining walls has a substantially flat side.

3. The retractor of claim 1, wherein the first and second retaining walls are nested relative to one another.

4. The retractor of claim 1, wherein at least one of the retaining walls has a curved bottom edge.

5. The retractor of claim 1, wherein at least one of the retaining walls has a compliant bottom edge.

6. The retractor of claim 1, further comprising a hinge that couples the first and second retaining walls.

7. The retractor of claim 1, further comprising a frame having a mechanism that holds the retaining walls apart from each other.

8. The retractor of claim 1, wherein the first guide receiving channel comprises a slot.

9. The retractor of claim 8, further including a second guide receiving channel, wherein both of the guide receiving channels are disposed in the frame.

10. The retractor of claim 9, wherein at least one of the guide receiving channels is slotted.

11. The retractor of claim 1, wherein at least a portion of the retractor is substantially transparent.

12. The retractor of claim 1, further comprising a web that couples distal portions of the retaining walls.

13. The retractor of claim 1, further comprising a plurality of removable finger processes extending from distal portions of the first retaining wall.

14. A retractor system comprising a retractor according to claim 1, and a first guide sized and dimensioned at one end to be received within the first guide receiving channel, and at another end to be inserted into a first area of bone.

15. The retractor system of claim 14, wherein the first guide is held in place with respect to the bone by a screw.

16. The retractor system of claim 14, further comprising a clamp or nut that cooperates with the first guide to assist in holding the frame in position relative to the bone.

17. The retractor system of claim 14, wherein the retractor has a second guide receiving channel spaced apart from the first guide receiving channel, and further comprising a second guide sized and dimensioned at one end to be received within the second guide receiving channel, and at another end to be inserted into a second area of bone.

18. The retractor system of claim 17, wherein the second guide is held in place with respect to the bone by a second screw.

19. The retractor system of claim 14, further comprising an expander having a handle and sloped walls.

20. A method of inserting a tissue retractor into a patient, comprising:
providing a retractor having paired tissue retracting surfaces and first and second guide receiving areas;
percutaneously implanting first and second guides into areas of different areas of bone in the patient;
then positioning upper ends of the first and second guides through the first and second guide receiving areas, respectively, inserting the retractor into tissue of the patient; and
moving the tissue retracting surfaces apart from one another.

21. The method of claim 20, wherein the step of implanting comprises screwing the first guide into a pedicle of a vertebra.

22. The method of claim 20, wherein the step of implanting comprises inserting the first and second guides into different bones.

23. The method of claim 20, further comprising stabilizing the retractor on the guides using a wire.
